(19) United States
(12) Patent Application Publication
(10) Pub. No.: US 2009/0198449 A1
(43) Pub. Date: Aug. 6, 2009

(54) Dual-Slot Biological Test Meter Test Procedure

(76) Inventors: Yao Ouyang, Janghe City (TW); Hsing Ouyang, Janghe City (TW); Ying Ouyang, Janghe City (TW)

Correspondence Address: Hung-Chih Tseng;HDLS IPR Services, PO Box 220746, Chantilly, VA, 20153, US

(21) Appl. No.: 12/419,196

(22) Filed: Apr. 6, 2009

Related U.S. Application Data
(63) Continuation-in-part of application No. 11/031,619, filed on Jan. 6, 2005.

(51) Int. Cl. G06F 19/00; G01N 33/48

(52) U.S. Cl. 702/19; 702/85

(57) ABSTRACT

A dual-slot biological test meter test procedure includes the steps of a) inserting a code card into a first test slot of a dual-slot biological test meter, b) inserting a test strip into a second test slot of the dual-slot biological test meter to initiate the dual-slot biological test meter; c) inputting predetermined parameters into the dual-slot biological test meter, d) judging if the result is over the threshold or not after application of the blood sample to the reaction zone of the inserted test strip, and then entering a first stage to electrically connect the two electrodes of the inserted test strip if the result is over the threshold, and then entering a second stage to output a voltage to the electrodes of the inserted test strip for a predetermined length of time set by the code card, e) entering a third stage to read the current value subject to the setting of the code card when the predetermined length of time is up, then f) calculating the test result through a computing method set by the code card subject to the current value obtained; and then g) showing the test result on a display.

### CROSS-REFERENCE TO RELATED APPLICATION

The present invention is a continuation-in-part of U.S. application Ser. No. 11/031,619 filed on Jan. 6, 2005, now pending.

### BACKGROUND OF THE INVENTION

1. Field of the Invention

The present invention relates generally to the test procedure of a biological test meter and more particularly, to a dual-slot biological test meter test procedure, which uses a code card to input parameters into the dual-slot biological test meter so that the dual-slot biological test meter can be used with different test strips from different suppliers.

2. Description of the Related Art

Various biological test meters have been developed for examining the concentration of glucose, cholesterol, and globubin in blood. During examination, the test strip is inserted into the meter, and then a drop of blood sample is dropped onto the reaction zone of the inserted test strip, and then the two electrodes at the reaction zone are electrically connected. After connection of electric current to the electrodes, the meter analyzes input signal, and therefore the concentration of glucose, cholesterol, and globubin in blood is obtained. However, different reagent suppliers are continuously developing different test strips. Regular biological test meters cannot fit all different test strips from different suppliers. One specific model of biological test meter may be used with specific test strips from one particular supplier. Further conventional biological test meters do not have the function of self-calibration. If a biological test meter is not well calibrated, the user cannot obtain the test result accurately.

Further, in FIG. 4 of U.S. Pat. No. 5,366,609, the circuit design of the biosensing meter adopts only a single voltage for testing a specific sample strip. It does not allow frequency modulation. The generated signal waveform is limited to a fixed mode.

### SUMMARY OF THE INVENTION

The present invention has been accomplished under the circumstances in view. It is the main object of the present invention to provide a dual-slot biological test meter test procedure, which enables a dual-slot biological test meter to use one test strip or two different test strips from different suppliers for measuring different test items. It is another object of the present invention to provide a dual-slot biological test meter test procedure, which provides a calibrate card for insertion into the dual-slot biological test meter to calibrate the linearity of the dual-slot biological test meter. It is still another object of the present invention to provide a dual-slot biological test meter test procedure, which automatically calculate the validity of the inserted test strip, refusing the use of an expired test strip. It is still another object of the present invention to provide a dual-slot biological test meter test procedure, which automatically judges the type of the inserted test strip.

To achieve these and other objects of the present invention, the dual-slot biological test meter test procedure comprises the steps of: a) inserting a code card into a first test slot of a dual-slot biological test meter to input predetermined parameters into the dual-slot biological test meter and then inserting a test strip into a second test slot of the dual-slot biological test meter to initiate the dual-slot biological test meter; b) judging if the result is over the threshold or not after application of the blood sample to the reaction zone of the inserted test strip, and then entering a first state to electrically connect the two electrodes of the inserted test, strip if the result is over the threshold, and then entering a second stage to output a voltage of a predetermined waveform or frequency to the electrodes of the inserted test strip for a predetermined length of time set by the code card; c) entering a third stage to read the current value subject to the settings of the code card when the predetermined length of time is up, and then to calculate the test result through a computing method set by the code card subject to the current value obtained, and then to show the test result on a display.

When compared to the single voltage method as shown in FIG. 4 of U.S. Pat. No. 5,366,609, the waveform according to the present invention shows a multistage voltage. Further, the invention allows frequency modulation. When the frequency is over 10K, the output waveform is shown in FIG. 6. When the frequency is below 10K, the output waveform shows an added waveform (see A1 in FIG. 7), and the signal is enhanced. This technique facilitates signal processing. Because the frequency of the multistage voltage is processed by the code card, the invention effectively removes noises. Further, because the signal is enhanced, it avoids interference of external noises and facilitates determination of test strip expiration.

Unlike the single slot design of U.S. Pat. No. 5,366,609, the dual-slot biological test meter provided subject to the present invention allows insertion of the code card and a test strip. Further, the dual-slot design of the present invention facilitates left-hand blood sampling as well as right-hand blood sampling. Further, unlike the ROM (read only memory) used in U.S. Pat. No. 5,366,609, the chip of the code card according to the present invention is an EEPROM (electronically erasable programmable read only memory) or EPROM (erasable programmable read only memory) practical for repeated use. Therefore, the invention is an environmentally friendly design. By means of the internal program in the meter, the internal data of the meter such as the meter's serial number or message code, the user's information and memory value can be filled in the code card, facilitating further user data processing by the supplier.

### BRIEF DESCRIPTION OF THE DRAWINGS

FIG. 1 illustrates the outer appearance of a dual-slot biological test meter according to the present invention.

FIG. 2 is a block diagram showing the control flow of the dual-slot biological test meter according to the present invention.

FIG. 3 is a circuit block diagram of the dual-slot biological test meter according to the present invention.

FIG. 4 illustrates the control waveform of the reference power supply according to the present invention.

FIG. 5 is a waveform chart showing a first test result reading method according to the present invention.

FIG. 6 is a waveform chart showing a second test result reading method according to the present invention.

FIG. 7 is a waveform chart showing a third test result reading method according to the present invention.

### DETAILED DESCRIPTION OF THE INVENTION

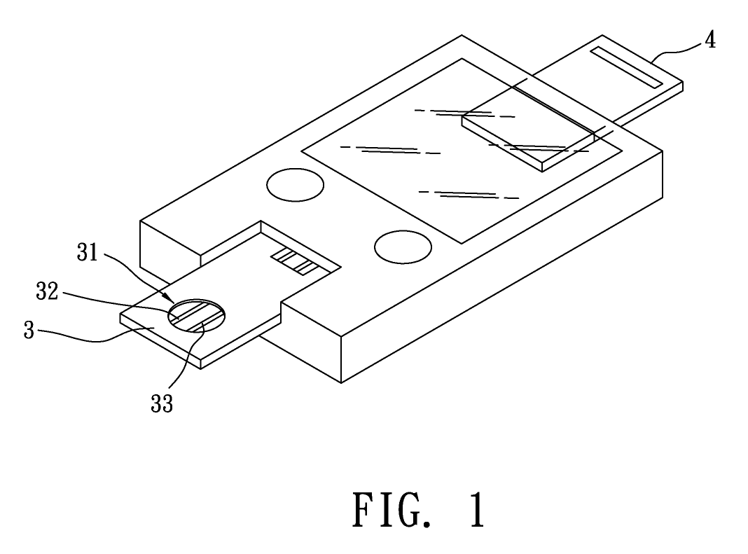

Referring to FIG. 1, a dual-slot biological test meter 1 is shown having two test slots 11,12 at two sides for receiving test strips 3 for examining a respective specific substance in the blood sample applied to each inserted test strip 3. This design of two test slots facilitates left-hand blood sampling as well as right-hand blood sampling. A code card 4 may be inserted into one of the test slots 11,12 to set the parameters of the dual-slot biological test meter 1, defining one test slot 11,12 to examine one specific type of test strip 3 only.

The dual-slot biological test meter 1 can provide a respective reference voltage to the inserted test strip 3 subject to the type of the inserted test strip 3. For example, the dual-slot biological test meter 1 can provide one single power source, multiple power sources, saw wave, or different frequencies subject to the parameters provided by the code card 4, i.e., the dual-slot biological test meter 1 fits different commercially available biological test strips.

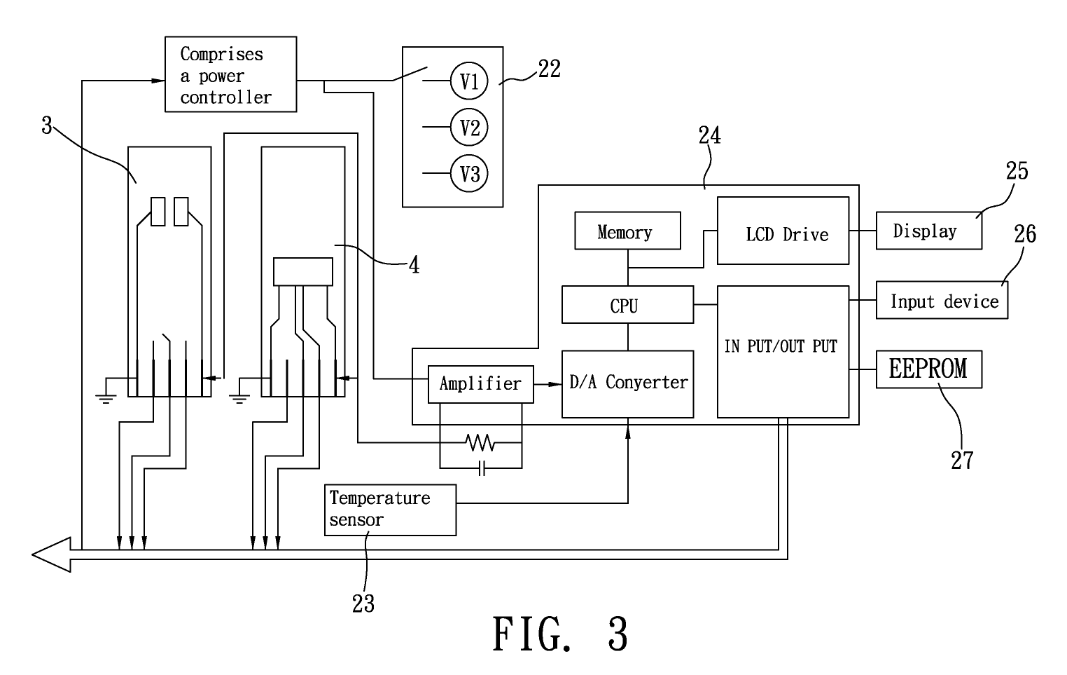

Referring to FIG. 3, the internal circuit 2 of the aforesaid dual-slot biological test meter 1 comprises a power controller 21, a reference voltage 22, a temperature sensor 23, a microprocessor 24, a display 25, an input device 26, and a first EEPROM (electronically erasable programmable read only memory) 27. The display 25, the input device 26, and the first EEPROM 27 are respectively connected to the microprocessor 24. In one embodiment, shown in FIG. 3, the microprocessor 24 includes an amplifier 241, a D/A converter 242, a CPU 243, a memory 244, an Input/Output control 245, and. a LCD drive 246. Because the construction of the individual components of the internal circuit 2 of the dual-slot biological test meter 1 is of the known art and not within the scope of the claims of the present invention, no further detailed description in this regard is necessary.

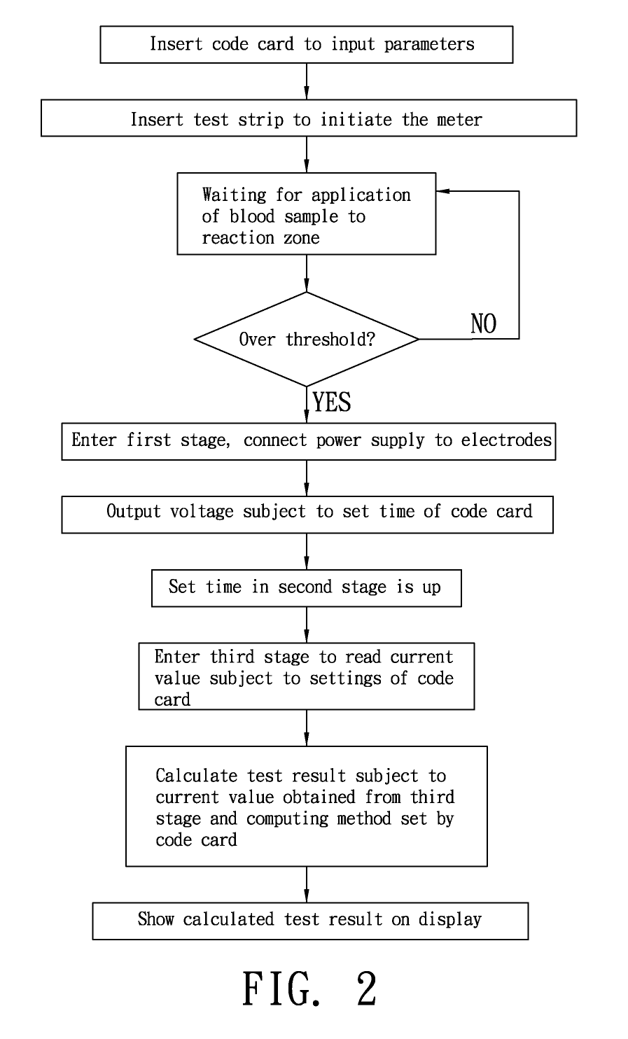

When in use, as shown in FIGS. 1-3, the code card 4 is inserted into one test slot 12 of the dual-slot biological test meter 1 to input parameters into the dual-slot biological test meter 1, and then the assigned test strip 3 is inserted into the other test slot 11 to initiate the dual-slot biological test meter 1. After blood sample has been applied to the reaction zone 31 of the inserted test strip 3, the dual-slot biological test meter 1 judges if over the threshold or not. If not, return to stand-by status. If the result is over the threshold, the dual-slot biological test meter 1 enters the first stage to electrically connect the two electrodes 32,33 of the inserted test strip 3 and then enters the second stage to output a voltage to the electrodes 32,33 for a predetermined length of time set by the code card 4. During the second state, the code card 4 provides a waveform or frequency subject to set voltage range. When the set time is up, the dual-slot biological test meter 1 enters the third stage to read the current value subject to the setting of the code card 4, and then to calculate the test result subject to the computing method set by the code card 4, and then to show the test result on the display 25.

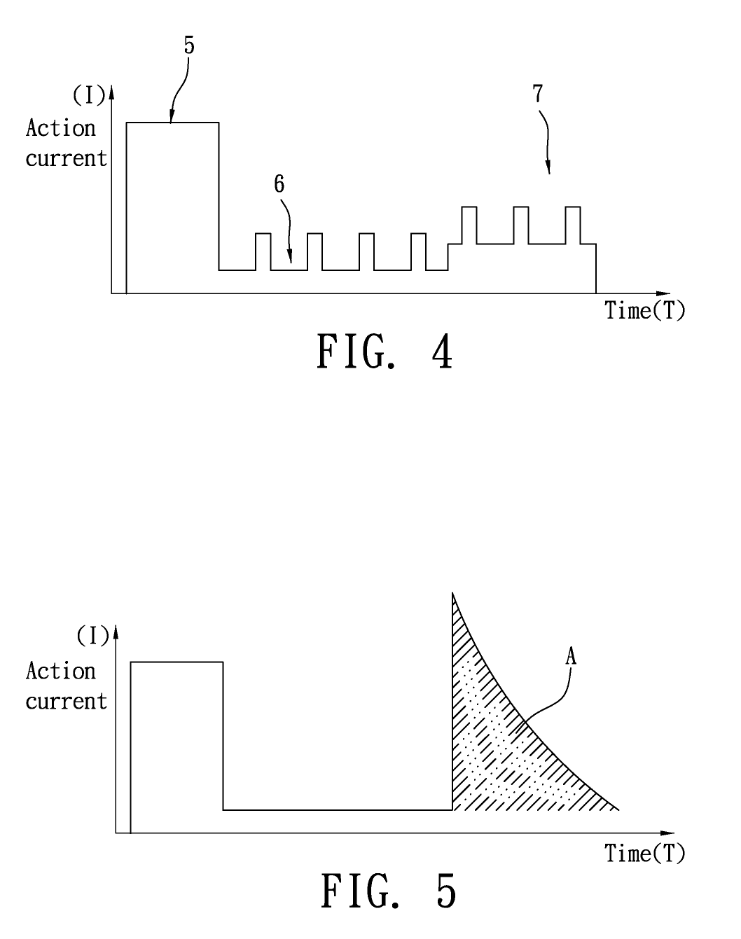

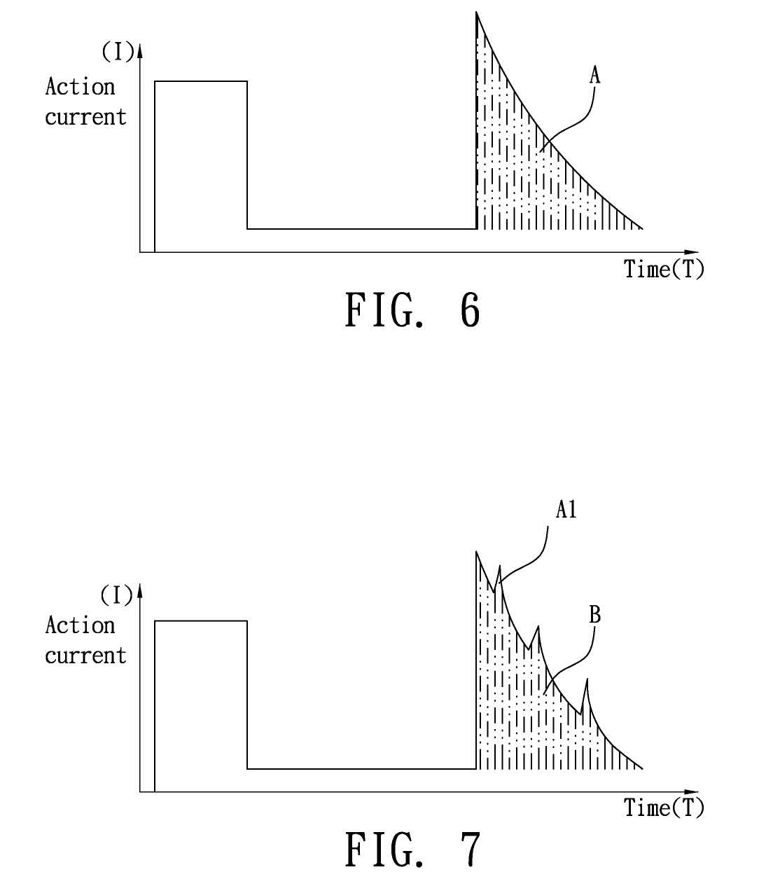

FIG. 4 illustrates the control waveform of the reference power supply. FIG. 5 illustrates the rest result waveform of the first method. FIG. 6 illustrates the test result waveform of the second method. As illustrated in FIG. 4, the control waveform of the reference power supply has three stages, namely, the first stage 5 to wait for receiving blood sample, the second stage 6 to waist for the reaction of the test strip 3, and the third stage 7 to read the result.

During the first stage 5, the two electrodes 32,33 of the test strip 3 are electrically connected (see FIG. 1), and the microprocessor 24 defines a reference voltage range and provides the signal to the power controller 21. At this time, the reference voltage 22 outputs a waveform or frequency to the two electrodes 32,33 subject to the control of the power controller 21. However, because the two electrodes 32,33 are in an open circuit status at this time, the current value measured by the dual-slot biological test meter 1 is below the threshold set in the code card 4. After dropping of blood sample to the reaction zone 31 of the test strip 3, the two electrodes 32,33 are closed; therefore the dual-slot biological test meter 1 enters the second stage 6.

When entered the second stage 6, a different reaction time is required for a different test item. For example, when examining blood glucose, the reaction time is about 2˜20 seconds; the reaction time is about 20˜180 seconds for cholesterol, or 20˜120 seconds for globubin. The predetermined reaction time for each test item is set in the code card 4 in factory subject to every commercially available type of test strip 3.

The third stage 7 begins immediately after the reaction time in the second stage 6 was up. The parameters for running the third stage 7 were set in the code card 4 in factory subject to every commercially available type of test strip 3. During this stage, the dual-slot biological test meter 1 provides a voltage ranging from 0 mV through 600 mV, however its voltage setting is below the voltage 0˜600 mV of the first stage 5. The electricity connection time varies with the test items, for example, the reaction time is about 2˜20 seconds for glucose, 2˜30 seconds for cholesterol, or 2˜60 seconds for globubin. These time settings were set in the EEPROM of the code card 4 in factory subject to every commercially available type of test strip 3.

When applying voltage during the third stage 7, the value of electric current measured between the two electrodes 32,33 of the inserted test strip 3 is obtained after reaction of the reagent of the inserted test strip 3 with the test item blood sample.

The test result can be read by means of two methods. Referring to FIG. 5, the first method is to read the test result by means of the integration formula of:

A(area)=∫di/dt

A1(area)=∫di/dt1

Meaning of A (area) and A1 (area): When a biological test strip is making a reaction, a waveform of area A is produced; Area A1 is a small waveform generated upon a multistage voltage; the area value is converted into a corresponding concentration value.

Meaning of i: When a biological test strip is making an oxidation-reduction reaction, a micro current is produced and gradually attenuated following running of time, thereby producing an area A, which is converted into a corresponding concentration value.

For example, if the concentration of glucose in blood measured is 50 mg/dL, 100 mg/dL, 150 mg/dL, or 200 mg/dL, the integration value shall be 5000, 10000, 15000, or 20000 respectively. By means of linear regression and when the frequency is above 10K, it is obtained that blood glucose concentration (mg/dL)=0.01×area A.

If the area obtained is 18000 when dropped one drop of blood sample of unknown concentration, the glucose concentration shall be 0.01×area A, i.e., 0.01×18000=180 mg/dL.

By means of linear regression and when the frequency is below 10K, it is obtained that blood glucose concentration (mg/dL)=0.01×area B.

If the area obtained is 18000 when dropped one drop of blood sample of unknown concentration, the glucose concentration shall be 0.01×area B, i.e., 0.01×18000=180 mg/dL.

Referring to FIG. 6, the second method is to obtain the test result by means of reading the reading of the test sample at a predetermined time interval and then adding up the readings and then calculating the test result through a compensation or conversion procedure. For example, as shown in FIGS. 6 and 7, if the glucose concentration is 50 mg/dL at each reading when set to read current value per every 0.5 second within the reaction time of 5 seconds, the total number of readings will be 9 (at 0.5 second, 1.0 second, 1.5 seconds, 2.0 seconds, 2.5 seconds, 3.0 seconds, 3.5 seconds, 4.0 seconds, 4.5 seconds, 5.0 seconds) and the sum of the readings will be 500; if the concentration is 100, 150, 200 mg/dL respectively, the sum will be 1000, 1500, 2000 respectively; by means of linear regression and when the frequency is over 10K, it is obtained that:

blood glucose concentration (mg/dL)=0.01×area A.

If the area of 1800 is obtained after application of a blood sample of unknown concentration to the inserted test strip, the concentration of glucose of the blood sample will be blood glucose concentration (mg/dL)=0.01×1800 (area A)=180 mg/dL.

By means of linear regression and when the frequency is below 10K, it is obtained that:

blood glucose concentration (mg/dL)=0.01×area B.

If the area of 1800 is obtained after application of a blood sample of unknown concentration to the inserted test strip, the concentration of glucose of the blood sample will be blood glucose concentration (mg/dL)=0.01×1800 (area B)=180 mg/dL.

The invention further provides a calibration function. When inserted a calibrate card into one test slot of the dual-slot biological test meter, the dual-slot biological test meter automatically calibrate the linearity, preventing an examination error due to the aged problem of one component part.

The invention also provides the function of automatically examine the validity of the inserted test strip, preventing an examination error due to the use of an expired test strip.

Further, after insertion of a test strip into the dual-slot biological test meter, the dual-slot biological test meter automatically judges the type of the inserted test strip subject to the parameters inputted by the inserted test strip.

Although a particular embodiment of the invention has been described in detail for purposes of illustration, various modifications and enhancements may be made without departing from the spirit and scope of the invention. Accordingly, the invention is not to be limited except as by the appended claims.

What is claimed is:

1. A dual-slot biological test meter test procedure comprising the steps of:
a) inserting a code card, having a non-volatile memory, into a first test slot of a dual-slot biological test meter; and then
b) inserting a test strip into a second test slot of said dual-slot biological test meter to initiate said dual-slot biological test meter;
c) inputting predetermined parameters from said non-volatile memory of said code card into said dual-slot biological test meter, said predetermined parameters providing auto-functions for increasing the reliability of test results including:
1) auto calibration of linearity of said test meter,
2) auto-reading a type of said inserted test strip,
3) auto-checking a validity of said inserted test strip and aborting test if said inserted test strip is past an expiration date,
4) setting test parameters specific to said inserted test strip,
5) setting the calculation method for test results specific to said inserted test strip;
d) judging if the result is over the threshold or not after application of the blood sample to the reaction zone of the inserted test strip, and then entering a first stage to electrically connect the two electrodes of the inserted test strip if the result is over the threshold, and then entering a second stage to output a voltage to the electrodes of the inserted test strip for a predetermined length of time set by said code card;
e) entering a third stage to read the current value subject to the setting of said code card when said predetermined length of time is up; then
f) calculating the test result through a computing method set by said code card subject to the current value obtained; and then
g) showing the test result on a display.

2. The dual-slot biological test meter test procedure as claimed in claim 1, wherein said dual-slot biological test meter comprises a power controller, a reference voltage, a temperature sensor, a microprocessor, a display, an input device, and a first EEPROM (electronically erasable programmable read only memory), said display and said input device and said first EEPROM being respectively electrically connected to said microprocessor.

3. The dual-slot biological test meter test procedure as claimed in claim 2, wherein said reference voltage is within 0˜600 mV.

4. The dual-slot biological test meter test procedure as claimed in claim 1, wherein said test strip comprises a reaction zone and two electrodes arranged in said reaction zone.

5. The dual-slot biological test meter test procedure as claimed in claim 1, wherein the reaction time for reaction of said test strip with the applied blood sample during said second state is about 2˜20 seconds for glucose, about 20˜180 seconds for cholesterol, about 20˜120 seconds for globubin.

6. The dual-slot biological test meter test procedure as claimed in claim 1, wherein the reaction time during said third stage is about 2˜20 seconds for glucose, about 2˜30 seconds for cholesterol, about 2˜60 seconds for globubin.

7. The dual-slot biological test meter test procedure as claimed in claim 5, wherein the reaction time during said second stage is set in said code card in factory subject to each type of test strip.

8. The dual-slot biological test meter test procedure as claimed in claim 6, wherein the reaction time during said third stage is set in said code card in factory subject to each type of test strip.

9. The dual-slot biological test meter test procedure as claimed in claim 1, wherein the test result is read by means of the integration formula of: A (area)=∫di/dt; concentration (mg/dL)=0.01×area (A).

10. The dual-slot biological test meter test procedure as claimed in claim 1, wherein the test result is read by means of reading the reading of the test sample at a predetermined time interval and then adding up the readings and then calculating the test result through a compensation or conversion procedure:
A(area)=∫di/dt; concentration(mg/dL)=0.1×area(A).

11. The dual-slot biological test meter test procedure as claimed in claim 1, further comprising the step of inserting a calibrate card into one of the two test slots of said dual-slot biological test meter to examine the linearity of said dual-slot biological test meter.

12. The dual-slot biological test meter test procedure as claimed in claim 1, wherein said dual-slot biological test meter has the function of automatically examining the validity of the inserted test strip.

13. The dual-slot biological test meter test procedure as claimed in claim 1, further comprising the step of automatically judging the type of the inserted test strip subject to the parameters inputted by the inserted test strip into said dual-slot biological test meter upon insertion of the test strip into said dual-slot biological test meter.

14. The dual-slot biological test meter test procedure as claimed in claim 1, wherein the test result is read by means of the integration formula of: A (area)=∫di/dt; A1 (area)=∫di/dt; concentration (mg/dL)=0.01×area (A).

15. The dual-slot biological test meter test procedure as claimed in claim 1, wherein the test result is read by means of reading the reading of the test sample at a predetermined time interval and then adding up the readings and then calculating the test result through a compensation or conversion procedure:
A(area)=∫di/dt; A1(area)=∫di/dt; concentration (mg/dL)=01×area(A).
